(19) United States
(12) Patent Application Publication
(10) Pub. No.: US 2025/0331805 A1
(43) Pub. Date: Oct. 30, 2025

(54) ULTRASOUND-BASED 3D LOCALIZATION OF FIDUCIAL MARKERS OR SOFT TISSUE LESIONS

(71) Applicant: Stryker Corporation, Portage, MI (US)

(72) Inventors: Ananth RAVI, Toronto (CA); Prashant PANDEY, Toronto (CA); John DILLON, Mississauga (CA)

(73) Assignee: Stryker Corporation, Portage, MI (US)

(21) Appl. No.: 18/866,477

(22) PCT Filed: May 19, 2023
(86) PCT No.: PCT/IB2023/000365

§ 371 Date: Nov. 15, 2024

Related U.S. Application Data
(60) Provisional application No. 63/343,571, filed on May 19, 2022.

(51) Int. Cl. A61B 8/08; A61B 8/00; A61B 90/00

(52) U.S. Cl. CPC A61B 8/0833; A61B 8/461; A61B 8/5223; A61B 90/39; A61B 2090/3925

(57) ABSTRACT

A method and system for localizing a target (e.g., a fiducial marker, or other target) in an individual. The method includes transmitting an ultrasonic signal from a transducer array. Radiofrequency (RF) signal data is generated based on a reflected signal received at the transducer array. The reflected signal results from the transmitted ultrasonic signal, and at least a portion of the reflected signal includes a signal reflected from the target. A location of the target is determined relative to the transducer array based on the RF signal data. The location may include a distance from the target to the transducer array and/or a direction of the target relative to the transducer array. A location indicator is provided to an operator. The location indicator is based on the determined location of the target.

### CROSS-REFERENCE TO RELATED APPLICATIONS

This application claims priority to U.S. Provisional Application No. 63/343,571, filed on May 19, 2022, now pending, the disclosure of which is incorporated herein by reference.

### FIELD OF THE DISCLOSURE

The present disclosure relates to localization of fiducial markers using ultrasound signals.

### BACKGROUND OF THE DISCLOSURE

To date, approaches for intraoperative localization have involved: implanted hooked wires and palpation, radioactive seeds and gamma detectors, infrared reflectors and custom probe, titanium implant and magnetic susceptometry, radiofrequency identification (RFID) implant and detector, electromagnetic (EM) coil and antenna, and MOLLI (magnetic fiducial and magnetic gradiometry). There continues to be a need for an alternate, wire-free non-radioactive approach to soft tissue lesion localization for surgical guidance and removal.

### BRIEF SUMMARY OF THE DISCLOSURE

In an aspect, the present disclosure may be embodied as a method for localizing a target (e.g., a fiducial marker, or other target) in an individual. The method includes transmitting an ultrasonic signal from a transducer array. Radiofrequency (RF) signal data is generated based on a reflected signal received at the transducer array. The reflected signal results from the transmitted ultrasonic signal, and at least a portion of the reflected signal includes a signal reflected from the target. A location of the target is determined relative to the transducer array based on the RF signal data. The location may include a distance from the target to the transducer array and/or a direction of the target relative to the transducer array. Determining a location of the target may include distinguishing the target from other artifacts in the RF signal data. A location indicator is provided to an operator. The location indicator is based on the determined location of the target.

In some embodiments, the location of the target is determined by feature extraction using a machine learning classifier on the RF signal data. For example, the location of the target may be determined directly from the RF signal data by feature extraction using a machine language classifier. The RF signal data may be preprocessed to increase a signal-to-noise ration of the RF signal data. The preprocessing may include, for example, transforming the RF signal data into frequency domain signal data; and applying one or more filters to the frequency domain signal data.

In some embodiments, the location of the target is determined using image processing of a B-mode image reconstructed from the RF signal data. Image processing may include, for example, analyzing the B-mode image to identify the target; and determining a distance from the target to the transducer array based on the identified target. In some embodiments, image processing may include determining a direction of the target relative to the transducer array based on the identified target. In some embodiments, analyzing the B-mode image includes image segmentation and/or classification.

The location indicator may be an audible tone and/or a visual display. For example, the location indicator may be provided by varying a pitch and/or volume of the audible tone according to the location of the target. In some embodiments, a visual display provides a visual representation of the distance from the target to the transducer array and/or the direction of the target relative to the transducer array.

In some embodiments, the method may be repeated. For example, the steps of transmitting an ultrasound signal and generating RF signal data are repeated. In this way, the location of the target may be updated.

The method may further include transmitting an additional ultrasonic signal from a transducer array; generating RF signal data based on a reflected signal received at the transducer array, the reflected signal resulting from the transmitted additional ultrasonic signal, wherein no portion of the reflected signal includes a signal reflected from the target; and identifying that no target is present in the reflected signal.

In another aspect, the present disclosure may be embodied as a system for localizing a target (e.g., fiducial marker, etc.) in an individual. Such a system includes a transducer array for transmitting and receiving ultrasound signals; and a processor in communication with the transducer array. The processor is programmed to cause the transducer array to transmit an ultrasonic signal; receive RF signal data from the transducer array, the RF signal data being based on a reflected signal received at the transducer array, wherein the reflected signal results from the transmitted ultrasonic signal, and wherein at least a portion of the reflected signal includes a signal reflected from the target; determine a location of the target relative to the transducer array based on the RF signal data; and provide a location indicator to an operator, the location indicator being based on the determined location of the target.

### DESCRIPTION OF THE DRAWINGS

For a fuller understanding of the nature and objects of the disclosure, reference should be made to the following detailed description taken in conjunction with the accompanying drawings, in which:

FIG. 1 is a diagram describing the identification of target (in this case, a fiducial marker) position and conversion to simplified feedback according to an embodiment of the present disclosure, via segmentation of B-mode images;

FIG. 2 is a diagram describing the identification of target (fiducial marker) position and conversion to simplified feedback according to another embodiment of the present disclosure, via filtering and feature analysis of raw RF signals received by the ultrasound probe; and

FIG. 3 is a chart according to another embodiment of the present disclosure.

### DETAILED DESCRIPTION OF THE DISCLOSURE

The present disclosure describes an approach that utilizes ultrasound to detect one or more inert markers that can be made out of plastic, metal, hydrogel or any other ultrasound visible material, and/or an anatomical target such as a mass. For convenience, the disclosure is described with reference to “targets” and/or “fiducial markers,” which should be broadly interpreted to describe a marker (e.g., an implantable marker, etc.), a region of interest (e.g., a tissue mass or any other anatomical target), an implant (e.g., orthopedic implant, etc.), or any other target. Conventional ultrasound can produce B-mode images using acoustic energy transmission and reflection principles. These images contain Raleigh noise and characteristic speckle patterns, making them challenging to interpret by users. In particular, surgeons are not experienced ultrasonographers and may not have the required expertise to interpret ultrasound images to reliably identify the markers or targets intraoperatively. Presently there are a number of sonography training programs to build this competence; however, the adoption of ultrasound-based intraoperative guidance is limited to <5% of surgeons. Instead, they rely on other localization modalities which can provide numerical, auditory, or graphical feedback, much like the MOLLI system offers.

In various embodiments, the present disclosure provides a method for automatically processing raw ultrasound radio frequency (RF) data and/or B-mode ultrasound images, to provide non-imaging feedback such as distance measurement, target coordinates, a graphical depiction of the marker position relative to the probe, and an auditory cue. This can be accomplished via algorithmic approaches, such as conventional image segmentation techniques or machine learning approaches. As is known, an ultrasound transducer generates RF signal data based on ultrasound signals (reflected signals) received at the transducer. Some embodiments of the present disclosure utilize signal processing techniques to determine spatial information directly from the RF signal data—without first converting the RF signal data into an image (or image data).

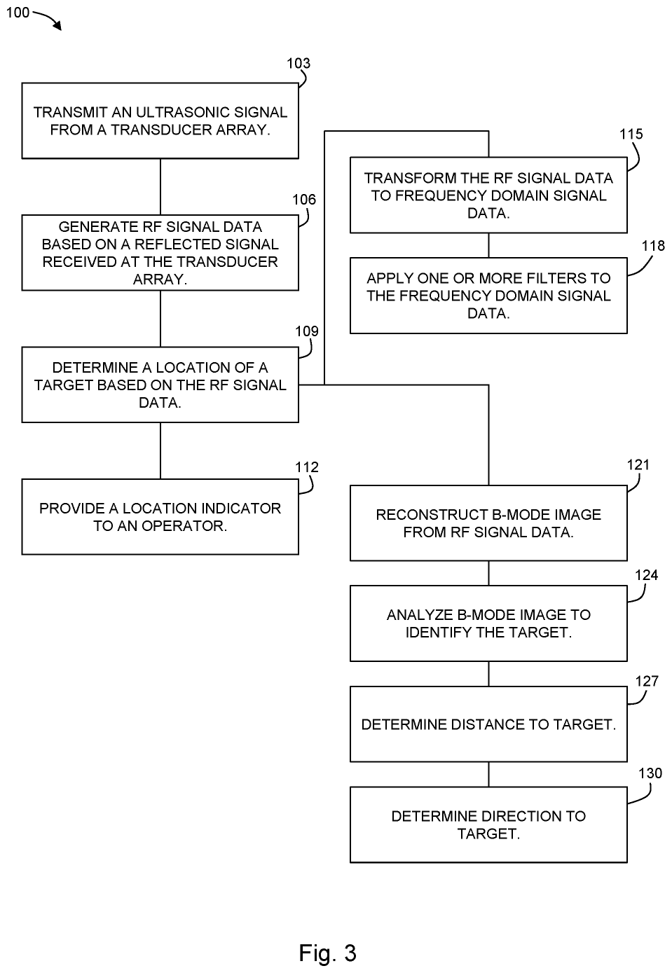

With reference to FIG. 3, in a first aspect, the present disclosure may be embodied as a method 100 for localizing a target (e.g., fiducial marker, region of interest, etc.) It should be noted that the target may include more than one targets—e.g., the method may be used to localize more than one target. The method 100 includes transmitting 103 an ultrasonic signal from a transducer array. The ultrasonic signal may be reflected back to the transducer array by body structures and tissues, implants, and the target. The transducer array generates 106 RF signal data based on the reflected signal—at least a portion of the reflected signal includes a signal reflected from the target when the target is in the field of view of the transducer array.

The method 100 includes determining 109 a location of the target relative to the transducer array based on the RF signal data. A location indicator is provided 112 to an operator based on the determined location of the target.

In some embodiments, the location of the target is determined using image-based techniques. In such embodiments, a B-mode image based on the RF signal data may be used.

In some embodiments, the location of the target is determined directly from the RF signal data—i.e., without the step of reconstructing an image from the RF signal data. For example, the location of the fiducial marker may be determined by feature extraction using a machine learning classifier, or frequency domain analysis. In the present disclosure, “direct” or “directly” from RF signal data is intended to describe that the RF signal data is processed to obtain a result without converting the RF signal data into image data. However, other processing of the RF signal data may occur (i.e., other than processing into image data) within the scope of such “direct” processing.

The RF signal data may be preprocessed to reduce noise. In some embodiments, the RF signal data may be transformed 115 into frequency domain signal data. Various techniques are known in the art for such transformation. For example, a Fourier transform may be used. One or more filters can then be applied 118 to the frequency domain signal data. For example, a low-pass filter may be applied to filter out high-frequency noise. Other filters—e.g., additional low-pass filters, high-pass filters, notch filters, etc.—may be applied as needed. The frequency-domain signal data may then be transformed back to the time domain for further processing.

In embodiments where image-based techniques are used, the method 100 may include reconstructing 121 a B-mode image from the RF signal data. The B-mode image may then be analyzed 124 to identify the fiducial marker(s). For example, image segmentation and/or classification techniques may be used to determine the image pixels corresponding to the target (and potentially other structures as well). Distance measurements from the target to the transducer array can then be determined 127. In some embodiments, a direction of the target relative to the transducer array is determined 130.

Whether an imaged-based approach or a direct approach based on the RF signal data is used, machine learning techniques may be used to identify the target and/or determine its location. For example, a machine-learning classifier may be used to identify the target from the background. In some embodiments, a machine-learning classifier can also distinguish the target from other artifacts (e.g., clips, implants, anatomical features, etc.) Machine learning classifiers may include artificial neural networks, such as, for example, convolutional neural networks (CNN), deep learning networks, etc.; support vector machines; and the like, or combinations of such techniques. Such classifiers may be trained on data sets of RF signal data or image data (as applicable) having known targets and locations.

In various embodiments, the location of the target includes a distance from the fiducial marker to the transducer array and/or a direction of the fiducial marker relative to the transducer array. The location indicator of the method may be a readily understandable—e.g., by personnel without specific training in ultrasound image interpretation. In some embodiments, the location indicator is an audible tone. For example, an audible tone may change in pitch and/or amplitude based on the location of the fiducial marker (i.e., location and/or direction). In some embodiments, the location indicator is a visual display. For example, an LCD monitor may provide a visual representation of the location of the fiducial marker. In an example embodiment, a distance from the fiducial marker may be indicated by a circle or an ellipse which decreases in diameter as the distance to the fiducial marker decreases. In some embodiments, more than one type of location indicator may be provided-for example, both audible and visual indicators, etc.

Embodiments of the disclosure include systems and methods for acquiring and processing ultrasound data and for presenting a graphical user interface that represents the position of the target (anatomy or marker) relative to the ultrasound transducer array.

Embodiments of the disclosed methods and systems are suitable for any application where there is a need to detect fiducial markers placed in human soft-tissue in a radiation-free manner that is not affected by nearby metal structures and electromechanical devices. It is particularly useful in scenarios where the clinical team does not require the additional diagnostic information that ultrasound images provide to achieve a successful therapeutic goal. Such use cases may include guidance for the excision/ablation of soft tissue lesions such as in breast, liver, lymph nodes, pancreas. The inert marker will be preoperatively placed inside the region-of-interest for each of these applications under radiographic, ultrasound, or magnetic resonance image guidance. In various embodiments, the presently-disclosed system or method will automatically process the ultrasound data and provide real-time feedback to the operator on the relative position of the marker to the ultrasound probe.

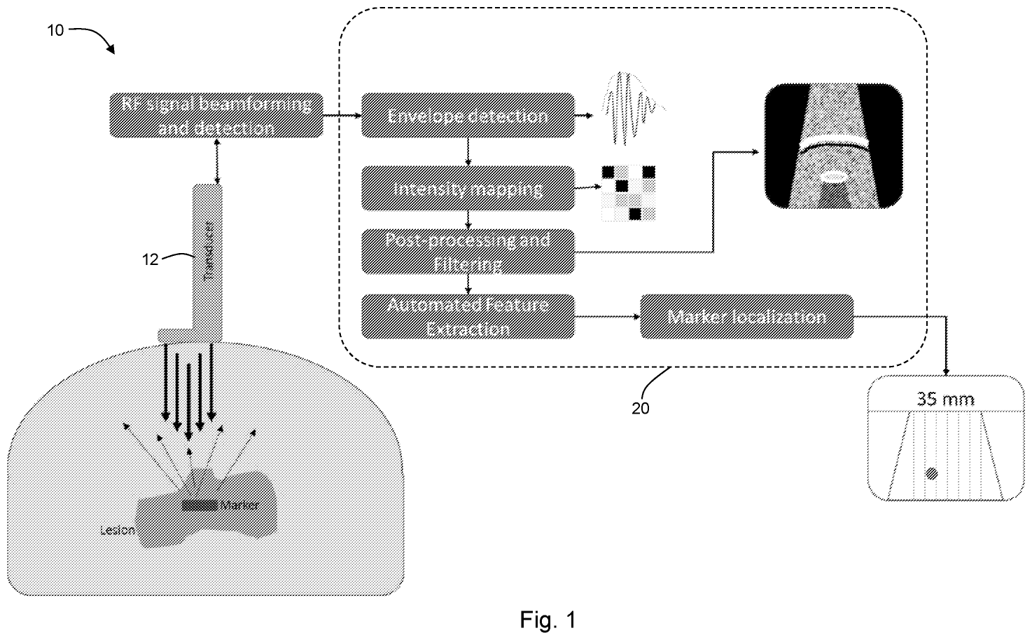

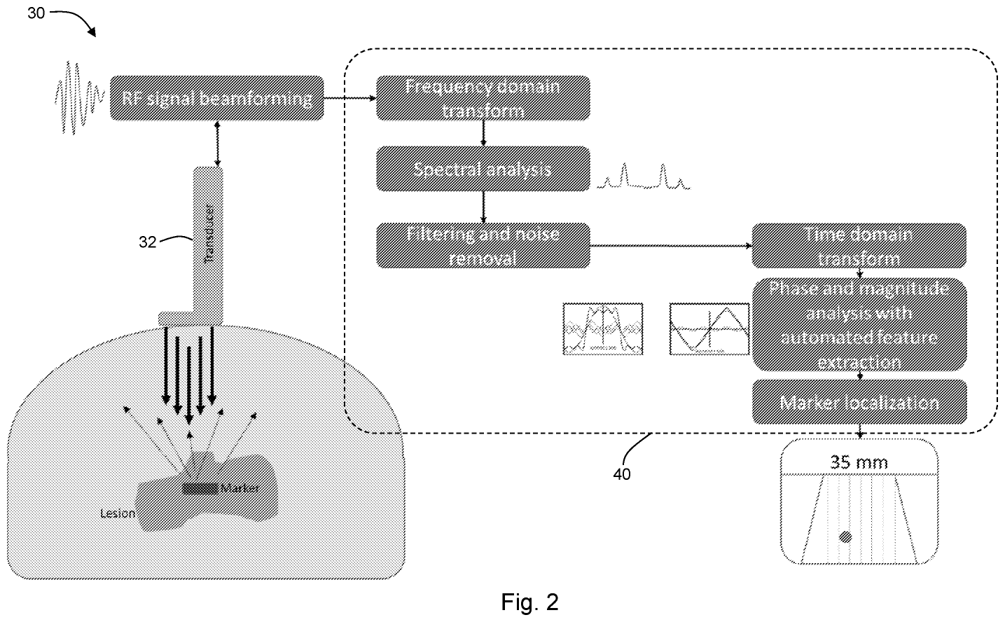

With references to FIGS. 1 and 2, in another aspect, the present disclosure may be embodied as a system for localizing a target (e.g., fiducial marker, etc.) in an individual. Such a system 10,30 includes a transducer array 12,32 for transmitting and receiving ultrasound signals; and a processor 20,40 in communication with the transducer array 12,32. The processor 20,40 may be programmed to perform any of the methods disclosed herein. For example, the processor 20,40 may be programmed to cause the transducer array 12,32 to transmit an ultrasonic signal; receive RF signal data from the transducer array, the RF signal data being based on a reflected signal received at the transducer array, wherein the reflected signal results from the transmitted ultrasonic signal, and wherein at least a portion of the reflected signal includes a signal reflected from the target; determine a location of the target relative to the transducer array based on the RF signal data; and provide a location indicator to an operator, the location indicator being based on the determined location of the target.

The processor may be configured based on a fiducial marker. In other words, the processor may be configured to localize a fiducial marker as the target. As above, the processor may be used to localize more than one target. In other words, the target may include multiple targets.

In some embodiments, the location of the target includes a distance from the target to the transducer array and/or a direction of the target relative to the transducer array. In some embodiments, the processor being programmed to determine a location of the target includes the processor distinguishing the target from other artifacts in the RF signal data.

With reference to the system 30 of FIG. 2, in some embodiments, the processor 40 includes a machine-learning classifier, and the location of the target is determined by feature extraction using the machine learning classifier on the RF signal data.

The processor may be further programmed to preprocess the RF signal data to increase a signal-to-noise ratio of the RF signal data. For example, the processor may be configured to preprocess the RF signal data by transforming the RF signal data into frequency domain signal data; and applying one or more filters to the frequency domain signal data.

With reference to the system 10 of FIG. 1, in some embodiments, the processor 20 determines the location of the target using image processing of a B-mode image reconstructed from the RF signal data. For example, the processor may be configured to perform image processing by analyzing the B-mode image to identify the target; and determining a distance from the target to the transducer array based on the identified target.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., determine a location of a target, etc.) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

Although the present disclosure has been described with respect to one or more particular embodiments, it will be understood that other embodiments of the present disclosure may be made without departing from the spirit and scope of the present disclosure.

1. A method for localizing a target in an individual, comprising:
transmitting an ultrasonic signal from a transducer array;
generating radiofrequency (RF) signal data based on a reflected signal received at the transducer array, the reflected signal resulting from the transmitted ultrasonic signal, wherein at least a portion of the reflected signal includes a signal reflected from the target;
determining a location of the target relative to the transducer array based on the RF signal data; and
providing a location indicator to an operator, the location indicator being based on the determined location of the target.

2. The method of claim 1, wherein the target is a fiducial marker.

3. The method of claim 1, wherein the location of the target includes a distance from the target to the transducer array and/or a direction of the target relative to the transducer array.

4. The method of claim 1, wherein determining a location of the target includes distinguishing the target from other artifacts in the RF signal data.

5. The method of claim 1, wherein the location of the target is determined by feature extraction using a machine learning classifier on the RF signal data.

6. The method of claim 1, further comprising preprocessing the RF signal data to increase a signal-to-noise ratio of the RF signal data.

7. The method of claim 6, wherein the preprocessing comprises:
transforming the RF signal data into frequency domain signal data; and
applying one or more filters to the frequency domain signal data.

8. The method of claim 1, wherein the location of the target is determined using image processing of a B-mode image reconstructed from the RF signal data.

9. The method of claim 8, wherein the image processing comprises:
analyzing the B-mode image to identify the target; and
determining a distance from the target to the transducer array based on the identified target.

10. The method of claim 9, wherein the image processing further comprises determining a direction of the target relative to the transducer array based on the identified target.

11. The method of claim 9, wherein analyzing the B-mode image comprises image segmentation and/or classification.

12. The method of claim 1, wherein the location indicator is an audible tone and/or a visual display.

13. The method of claim 12, wherein a pitch and/or volume of the audible tone varies according to the location of the target.

14. The method of claim 12, wherein the visual display provides a visual representation of the distance from the target to the transducer array and/or the direction of the target relative to the transducer array.

15. The method of claim 1, wherein the steps of transmitting an ultrasound signal and generating RF signal data are repeated.

16. The method of claim 15, further comprising updating the location of the target.

17. The method of claim 1, further comprising:
transmitting an additional ultrasonic signal from a transducer array;
generating RF signal data based on a reflected signal received at the transducer array, the reflected signal resulting from the transmitted additional ultrasonic signal, wherein no portion of the reflected signal includes a signal reflected from the target; and
identifying that no target is present in the reflected signal.

18. A system for localizing a target in an individual, comprising:
a transducer array for transmitting and receiving ultrasound signals;
a processor in communication with the transducer array, wherein the processor is programmed to:
cause the transducer array to transmit an ultrasonic signal;
receive RF signal data from the transducer array, the RF signal data being based on a reflected signal received at the transducer array, wherein the reflected signal results from the transmitted ultrasonic signal, and wherein at least a portion of the reflected signal includes a signal reflected from the target;
determine a location of the target relative to the transducer array based on the RF signal data; and
provide a location indicator to an operator, the location indicator being based on the determined location of the target.

19. The system of claim 18, wherein the processor is configured based on a fiducial marker as the target.

20. The system of claim 18, wherein the location of the target includes a distance from the target to the transducer array and/or a direction of the target relative to the transducer array.

21. The system of claim 18, wherein the processor is programmed to determine a location of the target includes the processor distinguishing the target from other artifacts in the RF signal data.

22. The system of claim 18, wherein the processor includes a machine-learning classifier, and the location of the target is determined by feature extraction using the machine learning classifier on the RF signal data.

23. The system of claim 18, wherein the processor is further programmed to preprocess the RF signal data to increase a signal-to-noise ratio of the RF signal data.

24. The system of claim 23, wherein the preprocessing comprises:
transforming the RF signal data into frequency domain signal data; and
applying one or more filters to the frequency domain signal data.

25. The system of claim 18, wherein the processor determines the location of the target using image processing of a B-mode image reconstructed from the RF signal data.

26. The system of claim 25, wherein the image processing comprises:
analyzing the B-mode image to identify the target; and
determining a distance from the target to the transducer array based on the identified target.
